(19) United States
(12) Patent Application Publication
(10) Pub. No.: US 2024/0392269 A1
(43) Pub. Date: Nov. 28, 2024

(54) ENZYMES WITH IMPROVED THERMOSTABILITY FOR THE DEGRADATION OF PLASTIC PRODUCTS

(71) Applicant: Daniel Eduardo RODRÍGUEZ CHAMORRO, Ciudad de México (MX)

(72) Inventor: Daniel Eduardo RODRÍGUEZ CHAMORRO, Ciudad de México (MX)

(21) Appl. No.: 17/910,349

(22) PCT Filed: Mar. 2, 2021
(86) PCT No.: PCT/IB2021/051742

§ 371 Date: Sep. 8, 2022

(30) Foreign Application Priority Data
Mar. 9, 2020 (MX) MX/A/2020/002672

(51) Int. Cl. C12N 9/18; C08J 11/10

(52) U.S. Cl. CPC C12N 9/18; C08J 11/105; C12Y 301/01074

(57) ABSTRACT

The present invention is located in the field of biotechnology, it refers to the creation of recombinant enzymes with improved thermostability, from mutations made in the Thermobifida fusca cutinase. Likewise, the present invention refers to the use of recombinant enzymes in the degradation of aromatic and semi-aromatic polymers and their oligomers, such as plastic.

### TECHNICAL FIELD OF THE INVENTION

The present invention is in the field of biotechnology, specifically the present invention refers to the creation of recombinant enzymes with improved thermostability, with potential to degrade aromatic polyesters, semi-aromatic and their oligomers, and the associated processes to degradation.

### REFERENCE TO AN ELECTRONIC SEQUENCE LISTING

The contents of the electronic sequence listing (sequence.txt; Size: 16,606 bytes; and Date of Creation: Nov. 3, 2023) is herein incorporated by reference in its entirety.

### BACKGROUND OF THE INVENTION

Polyethylene terephthalate (PET) is a type of plastic widely used in beverage containers and textiles. Currently PET is a worldwide problem, due to being a particularly resistant material to biodegradation because of its high crystallinity and the aromatic nature of its molecules, for all of these it is considered non-biodegradable, causing its accumulation in the environment. Annually are consumed at a worldwide level seventy-two million tons of virgin PET resin, despite the huge efforts that have been made in matters of collection and recycling it continues increasing the demand year after year of virgin resin, causing that annually fifty-three million tons end up in the environment. The global tendency is to migrate to a circular economy, in the case of PET this complicates due to the type of mechanical recycling that is currently used, which cannot treat plastic of NO food grade or very polluted. Therefore, new and better recycling methods are needed such as chemical recycling that allow to have greater reincorporation of the material. From the different types of chemical recycling, enzymatic chemical recycling is the most sustainable, due to the low reaction conditions and the no use of solvents and other toxic or dangerous catalysts for the environment (Wei, R and Zimmermann, W., 2017).

Currently, diverse hydrolase enzymes have been used for PET degradation for example, cutinases, lipases, carboxylesterases, esterases, proteases which have demonstrated certain ability to degrade PET. Cutinases are versatile enzymes that are able to catalize, apart from the rupture of the ester bond of the cutine, hydrolysis reactions and in vitro synthesis of a wide variety of substrates. These catalytic attributes have a potential use in different industries, for example, in food, detergents, biodesel production, bioremediation. Cutinases have been reported from various fungus, plants, and bacteria; Thermobifida fusca, Fusarium solani, Pseudozyma jejuensis, Aspergillus oryzae. Those are the enzymes that have shown better activity to degrade PET. However, due to not being specific enzymes to join and degrade PET, the reaction times are too high making impracticable its industrial implementation.

There are different inventions in the state of the art that are related to the degradation of plastic products by means of using of enzymes, for example, the application number WO2015025861, it refers to technology whereby it is isolated and synthesized the enzyme named PETASE or PET HIDROLASE that comes from a new species identified as Ideonella Sachalinensis, which is able to degrade polyethylene terephthalate (PET), catalyzing the decomposition of PET to produce monomers of monohydroxyethyl teraphthalate (MHET), and a second enzyme MHETASE degrades these monomers in ethylene glycol and terephthalic acid, the basic constituents of PET. Nevertheless, due to its low structural stability and solubility, it makes difficult its implementation in an industrial process, derived from these disadvantages different enzymes and mutations have been generated trying to improve the enzyme.

In 2018 different variations of PETASE were made public, including a double distal mutation to the catalytic center, making it more similar to the T. fusca Cutinase enzyme, and managing to make more efficient the depolymerization (Austin, H, et al., 2018). For its part in 2019, researchers from Korea, developed a new variation of IsPETASE, trying to improve the thermal stability and its activity. In particular, the variation IsPETASE S121E/D186H/R280A was designed, however, it only managed to increase the Tm (melting temperature) eight grades. (Hyeoncheol, F., & In Jin, et al., C. 2019). The international application with publication number WO2019168811 it also describes a modification in the PETASE to elevate its degrading capability, the PETASE was mutated in two residues of the active site. The patent document CN108467857, describes a PET hydrolase enzyme with different mutations, the mutated enzyme theoretically has a better PET degradation. Document CN108588052, it also describes a mutation in the PETASE enzyme; a point mutation in the biding site of the substrates, with these mutations the hydrolytic activity is increased and a PET degradation is possible at moderate temperatures (30° C.).

However, these new variations and mutations would be impracticable to be able to take them on an industrial process, the used methods to degrade PET require high temperatures, usually above their Tg (glass-transition temperature) 70° C. Which becomes a technological challenge and a problem to be solved to have the enzymes with improved thermostability that are also economically profitable. On the other hand, application Nr US2019233803 describes a cutinase derived from metagenome LC (Sulaiman, S, et al., 2012) with improved thermostability compared with commonly used cutinases to degrade polyethylene terephthalate. However, it has not been able to carry out a process on industrial scales. Since, it is not a specific enzyme, making its specificity constant low. In view of the disadvantages of the prior art, it is necessary to have new enzymes (specific and thermostable) and processes for the degradation of plastic products such as aromatic polyester and its oligomers.

### SUMMARY OF THE INVENTION

It is therefore an objective of the present invention to provide recombinant enzymes with different mutations and improved thermostability and degradation rate of aromatic polymers, semi-aromatic, and its oligomers, such as PET; the new enzymes present in the invention can be used in industrial processes for the degradation for example, beverage bottles, as well as other containers and textiles. In an additional aspect of the present invention, the recombinant enzymes present mutations, to increase thermostability and elevate its degradation capability of aromatic polymers, semi-aromatic and its oligomers.

In another embodiment the mutations of the present invention, refer, in a preferential aspect, to mutations conducted to the cutinase of Thermobifida fusca (T. fusca), where it has been located the active site of PETASE from Ideonella sachalinensis (I. sachalinensis), creating an enzyme similar to cutinase from T. fusca but with the catalytic activity of a PETASE from I. sachalinensis, in which we have an enzyme 93.1% similar to T. fusca with the catalytic characteristics of PETASE from I. sachalinensis.

In another embodiment of the invention, the residues H129 and F209 of the cutinase from T. fusca were modified, causing an accessibility of the second fraction of MHET of the PET chain. In a further embodiment of the invention, the cutinase from T. fusca has an extension in the connector loop of the region § 8-6, thanks to the inclusion of three residues N215, S216, N217; which allows the formation of a continuous cavity to join the long chains of PET.

In another embodiment of the invention, it has been integrated a disulfide bridge in the cutinase from T. fusca close to its active site favoring the increase of the degraded activity. In another embodiment of the invention, it has been modified region W155, adjacent to the catalytic site, of T. fusca, where the surrounding amino acids were modified switching from histidine H184 for S185.

In another embodiment of the invention, it was carried out a genetic re-engineering in the active site of cutinase from T. fusca; making ten mutations L203I, A206G, T207S, F209S, A210C, P211A, I213S, P214G, K216S, I217L, as well as the insertion of asparagine, glutamine and alanine residues among residues K216 and I217.

In another embodiment of the invention, it was modified the region that goes from G172 to L175 of the cutinase from T. fusca, making four mutations G172A, A173C, D174E and L175N.

In another embodiment of the invention, it was made a mutation of residues H184S, L157S and K159T, of the cutinase from T. fusca increasing its activity.

A further embodiment of the invention describes mutations were made in the residues H129W, A125G and L152Q. With these twenty-three changes it was obtained the SEQ. ID Nr 1.

In another embodiment of the invention, it was incorporated a disulfide bridge in the union site to calcium, with which the enzyme thermostability was improved. This modification involved the mutation of residues D204C and E253C, over the SEQ ID Nr 1, leading to SEQ ID Nr 2.

In another embodiment it was made the integration of five salt bridges to SEQ ID Nr 2; ARG 108-ASP 53-, ARG 124-ASP 98, ARG 173-GLU 176, ARG 173-ASP 193, ARG 286-ASP 28. For the incorporation of the first salt bridge it was deleted the region R18 and were mutated residues S19D and G219S, leading to sequence SEQ ID Nr 3.

In another embodiment of the invention a second salt bridge was integrated, modifying regions I86N, T87S, T88R and G61A, T62D, E63N, A64S, leading to SEQ ID Nr 4.

It is another embodiment of the invention the integration of a third salt bridge, making a mutation in the region S140E, leading to SEQ ID Nr 5.

In another embodiment of the invention it was incorporated a fourth salt bridge, making mutations in the regions N157D, N159T, W160F, S162N, V163T, T164S; just as region S161 was deleted, leading to sequence SEQ ID Nr 6.

It is another embodiment of the invention the integration of a fifth salt bridge, it was modified the entire C-terminus region from proline 243 to serine 258, which involved the following modifications: P243N, G244V, P245N, L249P, F250T, E252R, Y256F, S258T, as well as the deletion of residues, R246, G248 and G251. Leading to SEQ ID Nr 7.

Lastly, it is also an embodiment of the invention an in vitro method for the production of recombinant enzymes with the sequences SEQ ID Nr 1, SEQ ID Nr 2, SEQ ID Nr 3, SEQ ID Nr 4, SEQ ID Nr 5, SEQ ID Nr 6 and SEQ ID Nr 7.

In another embodiment, the recombinant enzymes generated in the current invention possesses the same catalytic constant as PETASE from I. sachalinensis (Yoshida, S, et al., 2016), but with significant increases in the Tm.

### BRIEF DESCRIPTION OF THE DRAWINGS

FIG. 1 refers to the structural alignment of cutinase from T.fusca (PDB 4CG1) and the PETASE from I. sachalinensis (PDB: 5XH3), where the substrate HEMT and the active site that is preserved in both enzymes can be observed.

FIG. 2 refers to the structural alignment of cutinase from T.fusca (PDB 4CG1) and the PETASE from I. sachalinensis (PDB: 5XH3), where the substrate HEMT and the four structural differences near the active site that both enzymes present can be observed.

FIG. 3 refers to the structure of cutinase from T.fusca (PDB 4CG1) showing the 23 changes that were made to incorporate the four structural differences that make different the PETASE from I. sachalinensis from the cutinase from T. fusca.

FIG. 4 refers to the structural alignment of Seq1 and the cutinase from S. viridis (PDB: 5ZNO).

FIG. 5 refers to a structure of cutinase LC (PDB 4EB0) showing the five salt bridges that the enzyme has and that the cutinase from T. fusca does not have.

FIG. 6 refers to a structure of Seq7 where showing the thirty-one changes that were carried out to incorporate the five salt bridges that cutinase LC possesses and that cutinase from T. fusca does not.

### DETAILED DESCRIPTION AND BEST MODE OF IMPLEMENTATION

Some aspects of the current invention will be now described in detail also using reference to the attached drawings, in which are shown some, but not all, the advantages of the current invention. In fact, various embodiments of the invention can be expressed in many different ways and should not be interpreted as limited to the embodiments described herein; rather, these exemplary embodiments are provided so that this invention is complete and thorough, and it completely communicates the significance of the invention to subject experts. For example, unless noted otherwise, something that is described as first, second or similar should not be interpreted as a particular order. As it is used in the description and in the attached assertions, the singular forms “a, an, the”, include plural references unless the content clearly indicates otherwise.

### Definitions

PET: It is a linear polymer constituted of terephthalate and ethylene glycol acid, which are connected through an ester bond, consequently, it is named polyester.

Plastic: Plastics are typically polymers of high molecular weight from organic molecules. Usually they are synthesized from petroleum chemical distillates (petrochemicals). The most important plastics used are poly ethylene (PE), polypropylene (PP), polystyrene (PS), polyvinyl chloride (PVC), polyethylene terephthalate (PET) and polyurethane (PU).

Esterase: An esterase is a hydrolase enzyme that splits ester bonds into the corresponding alcohols and acids through a chemical reaction in which a water molecule is used, that is, by means of a hydrolysis.

Cutinase: A cutinase is an enzyme that hydrolysis the cutin, producing cutin monomers.

Cutin: The cutin is a macromolecule principal component of the cutine of terrestrial plants. It is a polymer formed by many fatty acids of a long chain, that are united one with the other by ester bonds, creating a rigid three-dimensional net.

Mutant/Variant: It is a variation in the amino acid sequence, this can be introduced by substitution, deletion, or insertion of one or more codons in the nucleic acid sequence that encodes the enzyme which gives as a result a change in the amino acid sequence of the enzyme.

Recombinant enzyme: The recombinant proteins are those that are obtained by expressing a clone gene in a specie or a cell line different from the original cell.

Vector: The term vector or expression vector represent the bond through which a DNA or RNA sequence can be inserted in a host cell, with the purpose of transforming the host and to promote the introduce sequence expression.

Melting temperature (Tm): It is defined as the point in which half of the population of the protein is properly folded and the other half is denatured. The Tm is associated with the capacity of a protein to endure high temperatures.

Improved thermostability: It refers to mutations in the amino acid sequence that confer an increase in the Tm of a protein.

Aromatic polyesters: A derived polyester from monomers in which all hydroxyl groups and carboxyl groups are bonded directly to aromatic groups.

MHET: Basic unit of PET polymer, it only contains one terephthalic acid molecule bonded with an ethylene glycol molecule through an ester bond.

HEMT: methylated MHET.

The current invention refers to the creation of recombinant enzymes of cutinase from Thermobifida fusca, by means of different mutations to improve thermostability and its activity to degrade aromatic polyesters, semi-aromatic and its oligomers. Specifically, the current invention refers to different mutations that were made about cutinase from T. fusca from esterase from I. sachalinensis. Comparisons at a structural level using the DALI server show that the structure of the PETASE from Ideonella is quite similar to the structures of cutinase from T. fusca KW3 (PDB 4CG1, Z score 42.4), S. viridis (PDB 4WFJ, Z-score 42.3) and T. alba (PDB 3VIS, Z-score 42.1) (Joo, S, et al., 2018). These structural homologous have been reported of having activity to degrade PET and show an identity percentage of 50%, however, when comparing the enzymatic activity it was noticed that PETASE from I. sachalinensis is 120 times better than cutinase from T. fusca to degrade a PET film and 380 times better to degrade BHET oligomer (Yoshida, S, et al; 2016).

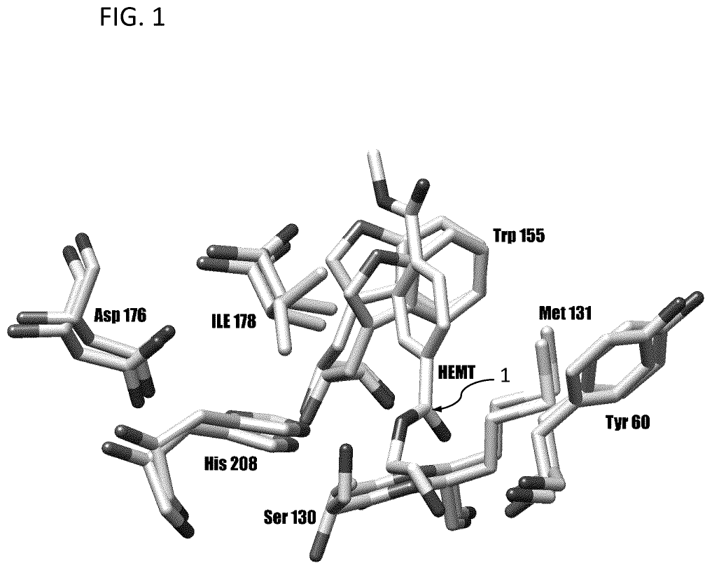

FIG. 1 shows the structural alignment of cutinase from T.fusca (PDB 4CG1) and the PETASE from I. sachalinensis (PDB: 5XH3), where the substrate HEMT 1 and the active site that is preserved in both enzymes can be observed. When making a structural alignment of PETASE from I. sachalinensis (PDB: 5XH3, the amino acid numbering is taken from this sequence) and the cutinase from T. fusca (PDB: 4CG1 the same numbering is taken) it can be observed that the 3 three residues that constitute the catalytic triad Si30, H208 and D176 of cutinase from T. fusca are located in the same position, indicating that the enzymes catalyze the degradation of PET through the same catalytic mechanism. The residues of Y60, M131, W155, 1178 of cutinase from T. fusca that constitute subsite 1 are also identical, which suggests that the union mode of the first MHET fraction is similar in both enzymes.

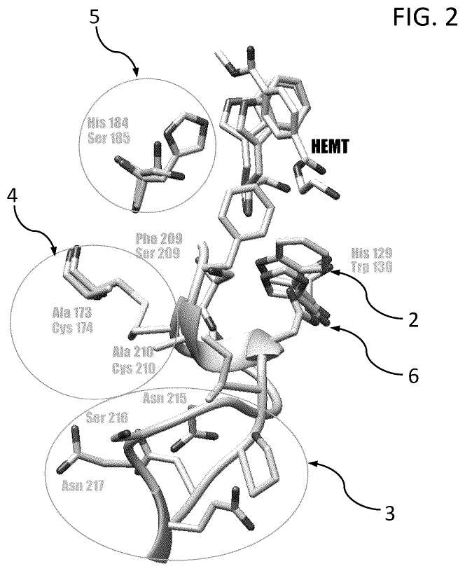

However, FIG. 2 shows four major structural differences between cutinase from T. fusca and the PETASE from I. sachalinensis. The first difference 2 it is found in subsite II. In the cutinase from T. fusca, the residues H129 and F209 are located in the correspondent positions of residues W130 and S209 of the PETASE from I. sachalinensis. The differences between these residues seem to make the subsite II of T. fusca narrower and deeper in comparison with the PETASE, causing a reduction in the accessibility of the second MHET section of the PET chain. The second difference 3 it is found in the connector loop of region β8-α6, where the PETASE possesses a more extended loop thanks to the presence of three extra residues N215, S216, N217. The unique formation of this extensive loop allows the PETASE to form a continuous cavity to join longer PET chains. Meanwhile in the cutinase from T. fusca this continuous cavity cannot be formed due to the obstruction of this loop. The third structural difference 4 is found in the presence of a unique disulfide bridge close to the active site formed by C174 and C210. In the cutinase from T. fusca and other PET degrading enzymes this bridge it is not found since instead of cysteines there are alanines in these positions. It has been noted that the presence of this disulfide bridge is fundamental for the activity of the PETASE since the activity decreases dramatically when mutated. The fourth difference 5 is found in the movement that is presented in residue W155. It has been noticed that the W155 adjacent to the catalytic site presents different configurations in the PETASE. Among adjacent amino acids it is found a S185 unique in the PETASE, since, in other counterparts, including cutinase from T. fusca, a histidine is always found in that location. Subsequent analyses have indicated that the H184 is remarkably close to the tryptophan obstructing its movement and affecting its activity. In FIG. 2 there can be observed the four structural differences close to the active site of both enzymes.

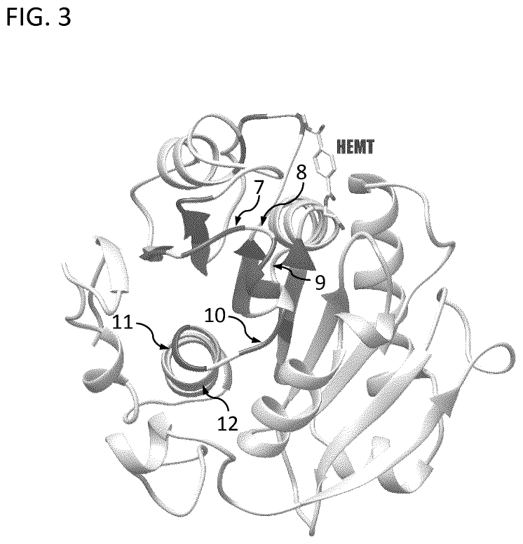

FIG. 3 shows the structure of cutinase from T.fusca (PDB 4CG1) with the twenty-three changes that were made to incorporate the four structural differences that make different the PETASE from I. sachalinensis from the cutinase from T. fusca. On this matter, the recombinant enzyme subject of the current invention refers, in a preferential aspect, to mutations made to the cutinase from T. fusca so that incorporates the four unique characteristics that are found in PETASE from I. sachalinensis, without interfering with other important structural interactions. The current invention consisted in twenty-three mutations that were made about cutinase from T. fusca, in order to incorporate the active site of PETASE from I. sachalinensis, for that purpose, it was made a complete reengineering of the active site, firstly, it was modified the region from L203 to I217. Ten mutations were made L203I, A206G, T207S (7), F209S (8), A210C and P211A (9), I213S and P214G(10), K216S and I217L (11), just as were inserted the asparagine residues, glutamine, and alanine among the K216 and I217 residues (12). Besides, it was modified the region that goes from G172 to L175, from T. fusca making four mutations G172A, A173C, D174E and L175N. There were also mutated residues H184S, L157S and K159T. Lastly, mutations were made in H129W, A125G and L152Q residues.

With these twenty-three changes between mutations and insertions, it was achieved to incorporate the four structural differences that make the PETASE from I. sachalinensis different from cutinase from T. fusca (FIG. 3), it means that it was achieved to modify residues H129 and F209, to extend on the connector loop of region β8-α6, to add the disulfide bridge, as well as to incorporate S184. In this manner, it was achieved the design of a new enzyme with thermophilic properties, but with enzymatic activity of the PETASE and which has the SEQ ID Nr 1.

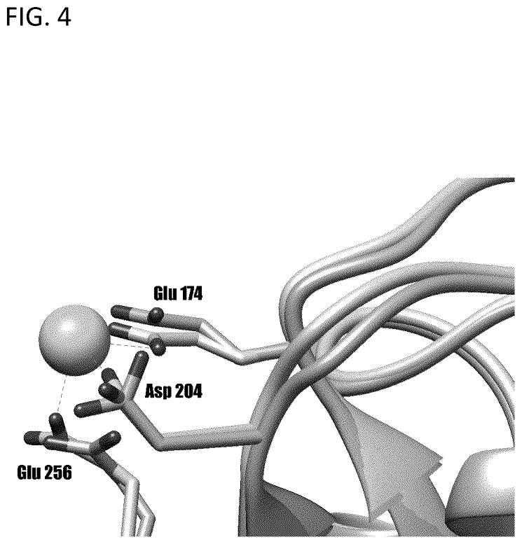

FIG. 4 refers to the structural alignment of Seq1 and the cutinase from S. viridis (PDB: 5ZNO). It can be observed the union site to calcium formed by residues Glu 220, Asp 250 and Glu 296, that the enzyme of the current invention possesses the very same residues in those positions. The previous art shows that the substitution of Asp 204 and Glu 256 for cysteines, forms a disulfide bridge that improves the thermostability of the enzyme between 6 and 9°.

It is another object of the invention the incorporation of another disulfide bridge in the biding site to calcium, that improves the thermostability of the enzyme increasing the Tm between 6 and 9° C. This modification involved the mutation of residues D204C and E256C on the SEQ ID Nr 1, leading to the SEQ ID Nr 2. With the purpose of making even more thermostable the enzyme, it was studied the cutinase derived from metagenome LC. This LC cutinase is the most thermostable cutinase reported up to the present, with a Tm of 86.2° C. on its own and of 94.6° C. in presence of calcium.

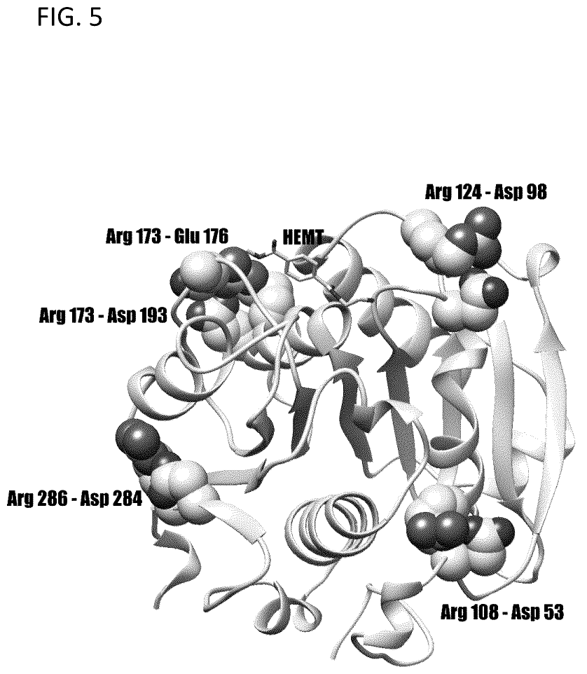

FIG. 5 compares the crystallographic structure of the LC cutinase (PDB:4EB0 and its numbering) and from the cutinase from T. fusca, there were found five salt bridges that possesses the LC cutinase and that the cutinase from T. fusca does not these are:
1.—ARG 108-ASP 53
2.—ARG 124-ASP 98
3.—ARG 173-GLU 176
4.—ARG 173-ASP 193
5.—ARG 286-ASP 284

It also shows the incorporation of these five salt bridges to the SEQ ID Nr 2. For the incorporation of the first salt bridge was deleted the R18 and the residues S19D and G219S were mutated. Leading to the SEQ ID Nr 3. The incorporation of the second salt bridge required the modification of I86N, T87S, T88R and G61A, T62D, E63N, A64S on the SEQ ID Nr 3 leading to the SEQ ID Nr 4. The incorporation of the third salt bridge required the mutation S140E on the SEQ ID Nr 4 to form the SEQ ID Nr 5. The incorporation of the fourth salt bridge required the following mutations N157D, N159T, W160F, S162N, V163T, T164S on the SEQ ID Nr 5, also, it was deleted S161 leading to the SEQ ID Nr 6.

Lastly, for the fifth salt bridge, even though cutinase from T. fusca has arginine 286 and aspartic 284, the structure shows that they do not form the salt bridge due to not being found in the appropriate conformation. This is due to the fact that the C-terminal of the cutinase presents a high mobility, which could affect the global stability of the enzyme. Consequently it was modified the whole region from proline 243 up to serine 258, which involves the following modifications: P243N, G244V, P245N, L249P, F250T, E252R, Y256F, S258T, as well as the deletion of residues R246, G248 and G251, leading to the SEQ ID Nr 7.

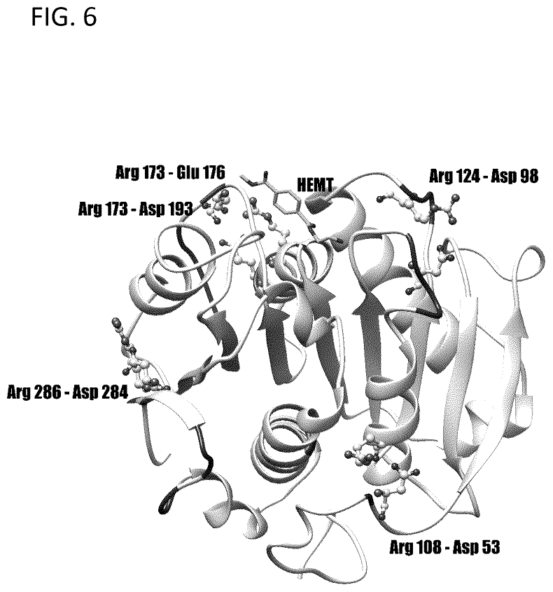

FIG. 6 shows thirty-one changes between mutations, deletions, and insertions to incorporate the five salt bridges from the LC cutinase to the T. fusca. The incorporation of these salt bridges is capable of increasing the thermostability of the enzyme gradually.

### Examples

The following examples are used in a merely illustrative way and do not have the intention of limiting the importance of the current invention. Some representative embodiments, as well as aspects that explain the advantages of the current invention are shown next.

### Example 1—Enzyme Obtaining Process

In particular, the current invention is related to various methods to produce recombinant enzymes that include: A) A DNA sequence that encodes for the amino acid sequence previously described and a cassette or vector fit for the recombinant expression of proteins. B) A method for the recovery of the produced enzyme. The in vitro expression systems are well-known by experts in the field and are commercially available. The described sequences were synthesized by using a codon optimization for E. coli (but it could be any organism expression, the important thing is that it is the sequence of the protein and not the DNA), in any expression vector capable for the expression system, where the expression can be induced by any promoter and inducer.

The production method comprises A) Host cells where was previously inserted the coding sequence of the enzyme in an expression method capable for the cell. B) Enzyme recovery from cell culture. Host cells are cultured in a nutrient medium appropriate for the production of polypeptides, using well-known methods in the field. For example, cells can be cultured in an Erlenmeyer flask or in small and big bioreactors (either by methods of batch, fed-batch or solid-state fermentation) on a laboratory or industrial scale. If the esterase is excreted into the middle of the culture, the enzyme can be recovered directly from the supernatant culture. On the contrary, the enzyme can be recovered from the lysed cells or after permeabilization. The esterase can be purified by any known method in the field. For example, esterase can be recovered from the middle of the culture by conventional procedures, but not limited, such as, collection, centrifugation, filtering, extraction, dry pulverization, evaporation, or precipitation. Optionally, the enzyme can be partially or totally purified by a variety of known processes in the field, for example, but not limited, chromatography (Ion exchange, chromatography hydrophobic or gel filtration), electrophoresis procedures (e.g., isoelectric focusing, preparations) differential solubility, (ammonium sulfate precipitation) SDS-PAGE, or extraction to obtain mostly pure polypeptides.

The enzyme can be used in its pure form, alone or in combination with additional enzymes for degradation and/or recycling of material that contains polyester. The enzyme can be in a soluble or immobilized form. In particular, it can be joined to cell membranes or lipid vesicles or synthetic supports such as glass, plastic, polymers, filters, membranes, either in the way of beds, columns, patches and similar. Other object of the invention is to promote a composition that comprises the enzyme and the host cell of the invention. In the context of the invention. The term “composition” covers any type of compositions that comprises the enzyme of the invention. The composition can be liquid or dry, for example, in the form of powder or lyophilized. For example, the composition can comprise the enzyme or the recombinant cells encoding for the enzyme of the invention or extracts and optionally excipients or reagents.

Appropriate excipients commonly include sorbents, reagents to adjust pH, preservatives such as benzoate, sorbate or sodium ascorbate, preservatives, stabilizing agents such as starch, dextrin, Arabic gum, salts, sugars, sorbitol, trehalose, lactose, glycerol, polyethylene glycol, EDTA, reducing agents, amino acids, carriers as solvents or aqueous solutions or similar. The composition of the invention can be obtained by mixing the enzyme with one several excipients. A particular fulfillment, the enzyme of the invention is solubilized in an aqueous medium together with one or several excipients, especially excipients that contribute to stabilize or protect the polypeptide from degradation. For example, esterase can be solubilized in water, eventually, with additional components such as glycerol, sorbitol, dextrin, starch, glycol, salts. The resulting mixing can be dry to obtain powder. Methods to dry the aforementioned mixture are quite well-known among people related with the field which include and are not limited to: lyophilization, cold drying, spray-drier, supercritical drying, drainage evaporation, thin film evaporation, centrifugal evaporation, conveyor belt drying, fluidized bed dryer, drum dryer or any combination of these methods. A particular fulfillment, the composition of the invention comprises at least a recombinant cell expressing the enzyme, or an extract. An extract of the cell designates any obtained fraction of a cell, such as supernatant of cells, precipitate cell, cell wall, DNA extracts, enzymes or preparation of enzymes or any derived preparation of the cells by a physicist, chemical and/or enzymatic treatment which is essentially free of cells. Extracts preferred are enzymatic active extracts. The composition of the invention can comprise one or several recombinant cells.

### Example 2—Evaluation of Thermostability

The thermostabilities of the variants of the cutinase have been determined and compared to the thermostability of the cutinase from T. fusca. Differential scanning fluorimetry. The differential scanning fluorimetry was used to assess the thermostability of the different enzymes by determining the denaturation temperature (Td). Temperature in which half the population of the enzymes is found denatured. Samples of the protein were prepared in a concentration of 1 mg/ml in buffer Tris-HCl pH 8, 300 mM NaCl, 10 mM de CaCl and SYPRO Orange 1× dye. It was taken 1 ul of each enzyme (1 ug) together with 9 ul of the previously mentioned buffer and it was put in a 96 well PCR plate. The plate was sealed, centrifuged at 2000 RPM for 1 min at room temperature. The experiment was made in a real-time thermocycler StepOnePlus of Applied Biosystems using excitation filters at 450/490 and emission 560/580. The samples were heated from 25° C. to 99.99° C. at a rate of 1.1° C./min with fluorescence measurements every 0.3° C. Denaturation temperatures were determined by making an adjustment to the curve of the Boltzmann equation. Wild type proteins and their variants were compared based in their Td values. Due to its high reproducibility between experiments about the same enzyme from different batches, a change in the Td of 1° C. was considered significant to compare the variants. Values of Td correspond to the average of at least 3 measurements.

### Example 3—Evaluation of the PET Degradation

The activity testing consisted in making enzymatic hydrolysis of the substrate BHET in MHET. The experiment consisted in taking 10 ul from a stock of 10 mg/ml of enzyme (100 ug total) and incubated with 100 mg of BHET in a solution of 10 mM of buffer Tris-HCl pH 8, 100 mM NaCl, 10 mM of CaCl, final volume of 1.5 ml. The reaction proceeded at 37° C. for two hours, the reaction was stopped heating at 110° C. for 10 min. the reaction was monitored by TLC, where it is observed the disappearance of BHET y la appearance of MHET using silica plates (UV 254), the plates were developed in 80% chloroform 20% acetic acid. The hydrolytic activity was determined by comparing the amount of substrate that the different enzymes were able to hydrolyze in the same period of time. In the following Table 1. It can be observed a summary of the obtained results from the experimental tests made.

| | Td | Hydrolytic Activity |
|---|---|---|
| Enzyme | ° C. | BHET |
| PETASE I. sakaiensis | 46 | 120x |
| Cutinase T. fusca | 68 | 1x |
| Seq 1 | 72 | 120x |
| Seq 2 | 78 | 120x |
| Seq 3 | 80 | 120x |
| Seq 4 | 84 | 120x |
| Seq 5 | 87 | 120x |
| Seq 6 | 92 | 120x |
| Seq 7 | 99 | 120x |

### REFERENCES

Austin, H; Allen, M; Donohoe, B; Rorrer, N; Kearns, F; Silveira, R. (2018). Characterization and engineering of a plastic degrading aromatic polyesterase. Proceedings of the National Academy of Sciences of the United States of America. Vol 115 (19): E4350-E4357.
Hyeoncheol, F; In Jin, C; Seongjoon, J; Hogyun, S; Hye-Young, S; So Young, C; Sang Yup, L; Kyung-Jin, Kim, C. (2019). Rational Protein Engineering of Thermo-Stable PETASE from Ideonella sachalinensis for Highly Efficient PET Degradation. ACS Catalysis 2019 9 (4), 3519-3526.
Joo, S; Cho, I; Seo, H; Son, H; Sagong, H; Shin, T; Choi, S; Lee, S; Kim, K. (2018). Structural insight into molecular mechanism of poly(ethylene terephthalate) degradation. Nature Communications. Vol 9(1), p. 382.
Sulaiman, S; Yamato, S; Kanaya, E; Kim, J; Koga, Y; Takano, K; Kanaya, S. (2012). Isolation of a novel cutinase homolog with polyethylene terephthalate-degrading activity from leaf-branch compost by using a metagenomic approach. Applied Environmental Microbiology. Vol 78(5), p. 1556-1562.
Wei, R; Zimmermann, W. (2017) Biocatalysis as a green route for recycling the recalcitrant plastic polyethylene terephthalate. Microbial Biotechnology. Vol 10(6), p. 1302-1307.
Yoshida, S; Hiraga, K; Takehana, T; Taniguchi, I; Yamaji, H; Maeda, Y; Toyohara, K; Miyamoto, K; Kimura, Y; Oda, K. (2016). A bacterium that degrades and assimilates poly(ethylene terephthalate. Science. Vol. 351 (6278), p. 1196-1199.

The following is claimed:

1. A recombinant enzyme of the esterase group, cutinases and lipases having at least 70% sequence identity of SEQ ID Nr 1 and a thermostability value of 68° C. to 99° C.

2. The enzyme according to claim 1 further comprising:
a. modification of residues H129 and F209;
b. incorporation of residues N215, S216, N217 and extension in connector loop region B8-α6;
c. incorporation of a disulfide bridge; and
d. modification in residue W155, histidine H184 for S185.

3. The recombinant enzyme according to claim 2, further comprising:
a. mutations in regions L203I, A206G, T207S, F209S, A210C, P211A, I213S, P214G, K216S, I217L; and
b. insertion of residues asparagine, glutamine, and alanine among residues K216 and I217.

4. The recombinant enzyme according to claim 3, further comprising mutations: G172A, A173C, D174E and L175N.

5. The recombinant enzyme according to claim 4 further comprising mutations in residues H184S, L157S and K159T, H129W, A125G and L152Q.

6. The recombinant enzyme according to claim 5 wherein said enzyme is a cutinase.

7. The recombinant enzyme according to claim 6 further comprising a disulfide bridge in the biding site to calcium.

8. The recombinant enzyme according to claim 7 further comprising a mutation in residues D204C and E253C.

9. The recombinant enzyme according to claim 8, by having further comprising at least 80% of identity of sequence SEQ ID Nr 2.

10. The recombinant enzyme according to claim 9 wherein region R18 and mutations S19D y G219S are deleted for incorporating a salt bridge.

11. The recombinant enzyme according to claim 10, further comprising at least 80% of identity of sequence SEQ ID Nr 3.

12. The recombinant enzyme according to claim 11, further comprising a second salt bridge, where the regions I86N, T87S, T88R and G61A, T62D, E63N, A64S were modified.

13. The recombinant enzyme according to claim 12, further comprising at least 80% of identity of sequence SEQ ID Nr 4.

14. The recombinant enzyme according to claim 12, further comprising a third salt bridge, where it was mutated region S140E.

15. The recombinant enzyme according to claim 14, further comprising at least 80% of identity of sequence SEQ ID Nr 5.

16. The recombinant enzyme according to claim 14, further comprising a fourth salt bridge, where regions N157D, N159T, W160F, S162N, V163T, and T164S were mutated and region S161 was deleted.

17. The recombinant enzyme according to claim 16, further comprising at least 80% of identity of sequence SEQ ID Nr 6.

18. The recombinant enzyme according to claim 16, further comprising a fifth salt bridge, where regions P243N, G244V, P245N, L249P, F250T, E252R, Y256F, and S258T were mutated and R246, G248 and G251 were deleted.

19. The recombinant enzyme according to claim 18, further comprising at least 80% of identity of sequence SEQ ID Nr 7.

20. The recombinant enzyme according to claim 19 further comprising a Td from 68° C. to 99° C. and a hydrolytic activity BHET equal to PETASE from I sachalinensis.

21. An in vitro method to produce the recombinant enzyme of sequences SEQ ID Nr 1 to SEQ ID Nr 7, comprising the steps of:
a. culturing host cells where the coding sequence was previously inserted; and
b. recovering said enzyme.

22. The use of recombinant enzymes of sequences SEQ ID Nr 1 to SEQ ID Nr 7 for the degradation of aromatic polymers, semi-aromatics and its oligomers, such as plastics.


SEQUENCE LISTING (7 sequences)

SEQ ID NO: 1 - protein, 264 aa, Artificial Sequence
  Esterase
ANPYERGPNPTDALLEARSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLGVMGWSMGGGGSLRLASQRPDLKAAIPQTPWHSNTNWSSVTVPTLIIACENDTIAPVATSAKPFYNSLPSSISKAYLEIDGGSHSCANSGNSNQALIGKYSVAWLKRFVDNDTRYTQFLCPGPRDGLFGEVEEYRSTCPF

SEQ ID NO: 2 - protein, 264 aa, Artificial Sequence
  Esterase
ANPYERGPNPTDALLEARSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLGVMGWSMGGGGSLRLASQRPDLKAAIPQTPWHSNTNWSSVTVPTLIIACENDTIAPVATSAKPFYNSLPSSISKAYLEICGGSHSCANSGNSNQALIGKYSVAWLKRFVDNDTRYTQFLCPGPRDGLFGEVCEYRSTCPF

SEQ ID NO: 3 - protein, 263 aa, Artificial Sequence
  Esterase
ANPYERGPNPTDALLEADGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLGVMGWSMGGGGSLRLASQRPDLKAAIPQTPWHSNTNWSSVTVPTLIIACENDTIAPVATSAKPFYNSLPSSISKAYLEICGGSHSCANSGNSNQALISKYSVAWLKRFVDNDTRYTQFLCPGPRDGLFGEVCEYRSTCPF

SEQ ID NO: 4 - protein, 263 aa, Artificial Sequence
  Esterase
ANPYERGPNPTDALLEADGPFSVSEENVSRLSASGFGGGTIYYPRENNTYGAVAISPGYTADNSSIAWLGERIASHGFVVITIDTNSRLDQPDSRAEQLNAALNHMINRASSTVRSRIDSSRLGVMGWSMGGGGSLRLASQRPDLKAAIPQTPWHSNTNWSSVTVPTLIIACENDTIAPVATSAKPFYNSLPSSISKAYLEICGGSHSCANSGNSNQALISKYSVAWLKRFVDNDTRYTQFLCPGPRDGLFGEVCEYRSTCPF

SEQ ID NO: 5 - protein, 263 aa, Artificial Sequence
  Esterase
ANPYERGPNPTDALLEADGPFSVSEENVSRLSASGFGGGTIYYPRENNTYGAVAISPGYTADNSSIAWLGERIASHGFVVITIDTNSRLDQPDSRAEQLNAALNHMINRASSTVRSRIDSSRLGVMGWSMGGGGSLRLAEQRPDLKAAIPQTPWHSNTNWSSVTVPTLIIACENDTIAPVATSAKPFYNSLPSSISKAYLEICGGSHSCANSGNSNQALISKYSVAWLKRFVDNDTRYTQFLCPGPRDGLFGEVCEYRSTCPF

SEQ ID NO: 6 - protein, 262 aa, Artificial Sequences
  Esterase
ANPYERGPNPTDALLEADGPFSVSEENVSRLSASGFGGGTIYYPRENNTYGAVAISPGYTADNSSIAWLGERIASHGFVVITIDTNSRLDQPDSRAEQLNAALNHMINRASSTVRSRIDSSRLGVMGWSMGGGGSLRLAEQRPDLKAAIPQTPWHSDTTFNTSVPTLIIACENDTIAPVATSAKPFYNSLPSSISKAYLEICGGSHSCANSGNSNQALISKYSVAWLKRFVDNDTRYTQFLCPGPRDGLFGEVCEYRSTCPF

SEQ ID NO: 7 - protein, 259 aa, Artificial Sequence
  Esterase
ANPYERGPNPTDALLEADGPFSVSEENVSRLSASGFGGGTIYYPRENNTYGAVAISPGYTADNSSIAWLGERIASHGFVVITIDTNSRLDQPDSRAEQLNAALNHMINRASSTVRSRIDSSRLGVMGWSMGGGGSLRLAEQRPDLKAAIPQTPWHSDTTFNTSVPTLIIACENDTIAPVATSAKPFYNSLPSSISKAYLEICGGSHSCANSGNSNQALISKYSVAWLKRFVDNDTRYTQFLCNVNDPTRVCEFRTTCPF